(19) United States
(12) Patent Application Publication
(10) Pub. No.: US 2016/0122294 A1
(43) Pub. Date: May 5, 2016

(54) METHODS FOR CO-PRODUCTION OF ALKYLBENZENE AND AN OLEOCHEMICAL FROM NATURAL OILS

(71) Applicant: UOP LLC, Des Plaines, IL (US)

(72) Inventors: Daniel L. Ellig, Arlington Heights, IL (US); Andrea G. Bozzano, Northbrook, IL (US)

(21) Appl. No.: 14/527,592

(22) Filed: Oct. 29, 2014

(51) Int. Cl. C07C 303/00; C07C 5/327; C07C 2/66; C07C 1/20

(52) U.S. Cl. CPC C07C 303/00; C07C 1/20; C07C 5/327; C07C 2/66

(57) ABSTRACT

Embodiments of methods for co-production of linear alkylbenzene and oleochemicals from a natural oil are provided. An exemplary method includes fat splitting the natural oil to form a stream comprising free fatty chains. The method includes fractionating the stream of free fatty chains to separate a first portion of free fatty chains and a second portion of free fatty chains. Further, the method includes processing the first portion of free fatty chains to provide the alkylbenzene product and processing the second portion of free fatty chains to form the oleochemical products.

### TECHNICAL FIELD

The technical field generally relates to methods for co-production of alkylbenzene and oleochemicals, and more particularly relates to methods for producing renewable alkylbenzene and an oleochemical from natural oils.

### BACKGROUND

Linear alkylbenzenes are organic compounds with the formula C₆H₅CₙH₂ₙ₊₁. While n can have any practical value, current commercial use of alkylbenzenes requires that n lie between 10 and 16, or more specifically between 10 and 13, between 12 and 15, or between 12 and 13. These specific ranges are often required when the alkylbenzenes are used as intermediates in the production of surfactants for detergents. Because the surfactants created from alkylbenzenes are biodegradable, the production of alkylbenzenes has grown rapidly since their initial uses in detergent production in the 1960s.

While detergents made utilizing alkylbenzene-based surfactants are biodegradable, processes for creating alkylbenzenes are not based on renewable sources. Specifically, alkylbenzenes are typically produced from kerosene extracted from the earth. Due to the growing environmental concerns over fossil fuel extraction and economic concerns over exhausting fossil fuel deposits, there is support for using an alternate source for biodegradable surfactants in detergents and in other industries.

There is also an increasing demand for the use of bio-sourced and biodegradable products in other segments of the chemical industry. For example, demand is rising for oleochemicals, which are chemical compounds derived from oils or fats from animal, plant or fungus sources. Oleochemicals may be used in the form of fatty alcohols, fatty acids, glycerin, amines, and methyl esters. Regardless of form, oleochemicals typically exhibit low toxicity and are suitable for applications where toxicity is of importance. Use in surfactants, soaps, detergents, lubricants and other downstream renewable chemicals may further increase demand for oleochemicals.

Accordingly, it is desirable to identify new sources of linear alkylbenzenes and oleochemicals. Further, it is desirable to provide methods and systems that provide renewable alkylbenzenes and oleochemicals. Furthermore, other desirable features and characteristics will become apparent from the subsequent detailed description and the appended claims, when taken in conjunction with the accompanying drawing and this background.

### SUMMARY

Embodiments of methods for co-production of linear alkylbenzene and oleochemicals from a natural oil are provided. An exemplary method for co-production of an alkylbenzene product and an oleochemical product from a natural oil comprises fat splitting the natural oil to form a stream of free fatty chains. The method fractionates the stream of free fatty chains to separate a first portion of free fatty chains and a second portion of free fatty chains. The method includes processing the first portion of free fatty chains to provide the alkylbenzene product. Further, the method includes processing the second portion of free fatty chains to form the oleochemical product.

In another exemplary embodiment, a method is provided for co-production of an alkylbenzene product and an oleochemical product from natural oil source triglycerides. The method includes fat splitting the natural oil source triglycerides to form a stream comprising glycerol and fatty acids. The method includes fractionating the stream to separate a first portion of fatty acids and a second portion of fatty acids. The method deoxygenates the first portion of fatty acids to form normal paraffins, dehydrogenates the normal paraffins to provide mono-olefins, alkylates benzene with the mono-olefins under alkylation conditions to provide an alkylation effluent comprising alkylbenzenes and benzene, and isolates the alkylbenzenes to provide the alkylbenzene product. The method includes processing the second portion of fatty acids to form the oleochemical product.

In accordance with another embodiment, a method for co-production of an alkylbenzene product and an oleochemical product from a natural oil includes deoxygenating a first portion of fatty acids with hydrogen to form a stream comprising paraffins. The methods includes dehydrogenating the paraffins to provide mono-olefins and hydrogen, recycling the hydrogen to support deoxygenating the first portion of fatty acids; alkylating benzene with the mono-olefins under alkylation conditions to provide an alkylation effluent comprising alkylbenzenes and benzene; and isolating the alkylbenzenes to provide the alkylbenzene product. The method further includes processing a second portion of fatty acids to form the oleochemical product.

### BRIEF DESCRIPTION OF THE DRAWING

Embodiments of methods for co-production of alkylbenzene and oleochemical products from natural oils will hereinafter be described in conjunction with the following drawing FIGURE wherein:

FIG. 1 schematically illustrates an apparatus for co-production of alkylbenzene and an oleochemical in accordance with an exemplary embodiment.

### DETAILED DESCRIPTION

The following Detailed Description is merely exemplary in nature and is not intended to limit the methods for co-production of an alkylbenzene and an oleochemical from natural oils. Furthermore, there is no intention to be bound by any theory presented in the preceding Background or the following Detailed Description.

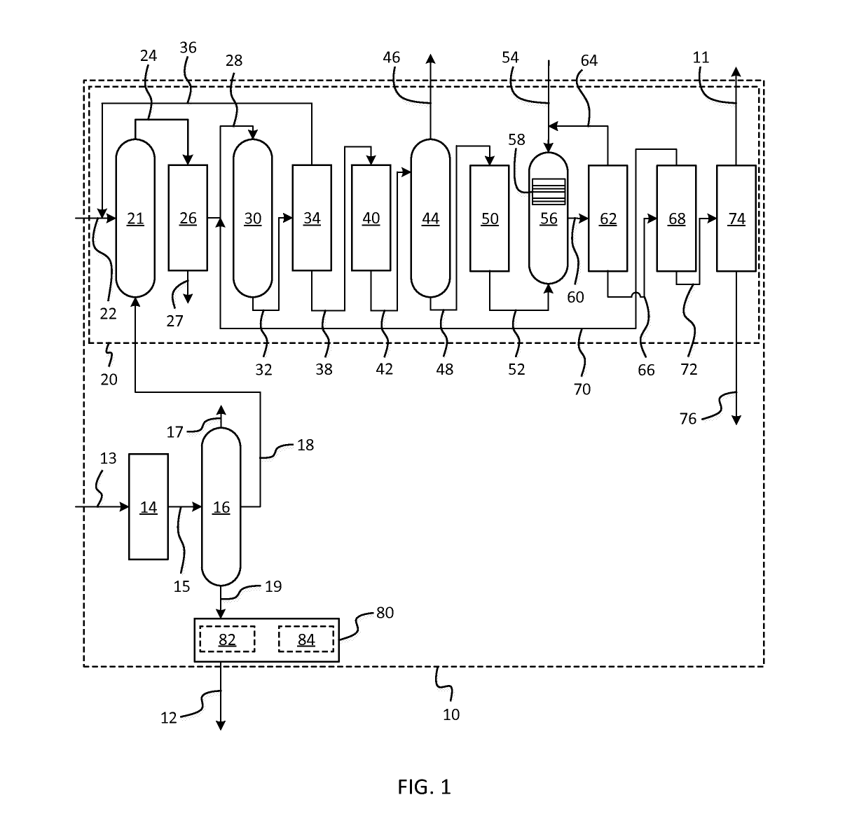

Various embodiments contemplated herein relate to methods and systems for co-production of an alkylbenzene and an oleochemical from natural oils. In FIG. 1, an exemplary apparatus 10 for producing an alkylbenzene 11 and an oleochemical 12 from a natural oil feed 13 is illustrated. As used herein, natural oils are those derived from animal, plant or fungal matter, and are often referred to as renewable oils. Natural oils are not based on kerosene or other fossil fuels. In certain embodiments, the natural oils include one or more of palm kernel oil, coconut oil, babassu oil, castor oil, cooking oil, and other vegetable, nut or seed oils. The natural oils typically comprise triglycerides, free fatty acids, or a combination of triglycerides and free fatty acids.

In the illustrated embodiment, the natural oil feed 13 is delivered to a fat splitting unit 14. In the fat splitting unit 14, the triglycerides are split into free fatty chains. Specifically, fat splitting occurs according to the equation: one mole triglyceride+3 moles water=one mole glycerol+3 moles of fatty acid. A stream of fatty chains and glycerol 15 is formed by the fat splitting unit 14 and is fed to a separator 16. The separator 16 may be a multi-stage fractionation unit, distillation system or similar known apparatus. In any event, the separator 16 separates a stream of glycerol 17, a first portion 18 of fatty chains and a second portion 19 of fatty chains. Exemplary embodiments may include a separator for removing glycerol from stream 15 before entering separator 16. In certain embodiments, the first portion of fatty chains 18 has carbon chain lengths of C10 to C14. In other embodiments, the first portion of fatty chains 18 has carbon chain lengths having a lower limit of CL, where L is an integer from four (4) to thirty-one (31), and an upper limit of CU, where U is an integer from five (5) to thirty-two (32). The second portion of fatty chains 19 may have carbon chains shorter than, longer than, or a combination of shorter and longer than, the chains of the first portion of fatty chains 18. In an exemplary embodiment, the first portion of fatty chains 18 comprises C10 to C13 fatty chains and the second portion of fatty chains 19 comprises fatty chains with C9− fatty chains, i.e., C9 and shorter chains, and C14+ fatty chains, i.e., C14 and longer chains. While shown as a single stream exiting the separator 16, in such an embodiment, the second portion of fatty chains 19 includes an upper or light draw of C9− chains and a lower or heavier draw of C14+ chains from the separator 16, while the first portion of fatty chains 18 would be taken as a side draw between the upper and lower draws.

An exemplary first portion of fatty chains 18 includes no more than about 2 weight percent (wt %) C9− fatty chains and no more than about 1 wt % C14+ fatty chains. Further, an exemplary first portion of fatty chains 18 includes at least about 97 wt % of C10 to C13 chains. C10 to C13 chains are particularly suited for the production of alkylbenzene, and the separation of C10 to C13 chains provides for efficient processing to form alkylbenzene and for the efficient processing of the remaining chains to form oleochemicals.

As shown in FIG. 1, the first portion of fatty chains 18 is introduced to an alkylbenzene production unit 20. Specifically, the first portion of fatty chains 18 is fed to a deoxygenation unit 21 which also receives a hydrogen feed 22. In the deoxygenation unit 21, the first portion of fatty chains 18 is deoxygenated and the fatty chains are converted into normal paraffins.

In FIG. 1, a deoxygenated stream 24 containing normal paraffins, water, carbon monoxide, carbon dioxide and propane exits the deoxygenation unit 21 and is fed to a separator 26. The separator 26 may be a multi-stage fractionation unit, distillation system or similar known apparatus. The separator 26 removes the water, carbon monoxide, carbon dioxide, and propane as stream 27 from the deoxygenated stream 24. While a single stream 27 is illustrated for simplicity, the water, carbon monoxide, carbon dioxide, and propane may be removed in separate streams. As shown, removal of the water, carbon monoxide, carbon dioxide, and propane by the separator 26 forms a normal paraffin stream 28. The normal paraffin stream 28 is fed to a dehydrogenation unit 30 in the alkylbenzene production unit 20. In the dehydrogenation unit 30, the normal paraffins are dehydrogenated into mono-olefins of the same carbon numbers as the paraffins. Typically, dehydrogenation occurs through known catalytic processes, such as the conventional Pacol process. Di-olefins (i.e., dienes) and aromatics are also produced as an undesired result of the dehydrogenation reactions.

In FIG. 1, a dehydrogenated stream 32 exits the dehydrogenation unit 30, and the dehydrogenated stream 32 comprises mono-olefins and hydrogen as well as some di-olefins and aromatics. The dehydrogenated stream 32 is delivered to a phase separator 34 for removing the hydrogen from the dehydrogenated stream 32. As shown, the hydrogen exits the phase separator 34 in a recycle stream of hydrogen 36 that can be added to the hydrogen feed 18 to support the deoxygenation process upstream.

At the phase separator 34, a liquid stream 38 is formed and comprises the mono-olefins as well as di-olefins and aromatics formed during dehydrogenation. The liquid stream 38 exits the phase separator 34 and enters a selective hydrogenation unit 40, such as a DeFine reactor. The hydrogenation unit 40 selectively hydrogenates at least a portion of the di-olefins in the liquid stream 38 to form additional mono-olefins. As a result, an enhanced stream 42 is formed with an increased mono-olefin concentration as compared to the liquid stream 38.

As shown, the enhanced stream 42 passes from the hydrogenation unit 40 to a lights separator 44, such as a stripper column, which removes a light end stream 46 containing any lights, such as butane, propane, ethane and methane, that resulted from cracking or other reactions during upstream processing. With the lights removed, stream 48 is formed and may be delivered to an aromatic removal apparatus 50 that removes aromatics from the stream 48 and forms a stream rich in mono-olefins 52. As referred to herein, “rich” means that the stream at issue includes at least 50 weight % of the referenced compounds.

In FIG. 1, the stream of mono-olefins 52 and a stream of benzene 54 are fed into an alkylation unit 56. The alkylation unit 56 holds a catalyst 58, such as a solid acid catalyst, that supports alkylation of the benzene 54 with the mono-olefins 52. Hydrogen fluoride (HF) and aluminum chloride (AlCl₃) are two major catalysts in commercial use for the alkylation of benzene with linear mono-olefins and may be used in the alkylation unit 56. As a result of alkylation, alkylbenzene, typically called linear alkylbenzene (LAB), is formed and is present in an alkylation effluent 60.

To optimize the alkylation process, surplus amounts of benzene 54 are supplied to the alkylation unit 56. Therefore, the alkylation effluent 60 exiting the alkylation unit 56 contains alkylbenzene and unreacted benzene. Further the alkylation effluent 60 may also include some unreacted paraffins. In FIG. 1, the alkylation effluent 60 is passed to a benzene separation unit 62, such as a fractionation column, for separating the unreacted benzene from the alkylation effluent 60. This unreacted benzene exits the benzene separation unit 62 in a benzene recycle stream 64 that is delivered back into the alkylation unit 56 to reduce the volume of fresh benzene needed in stream 54.

As shown, a benzene-stripped stream 66 exits the benzene separation unit 62 and enters a paraffinic separation unit 68, such as a fractionation column. In the paraffinic separation unit 68, unreacted paraffins are removed from the benzene-stripped stream 66 in a recycle paraffin stream 70, and are routed to and mixed with the normal paraffin stream 28 before dehydrogenation as described above.

Further, an alkylbenzene stream 72 is separated by the paraffinic separation unit 68 and is fed to an alkylate separation unit 74. The alkylate separation unit 74, which may be, for example, a multi-column fractionation system, separates a heavy alkylate bottoms stream 76 from the alkylbenzene stream 72.

As a result of the post-alkylation separation processes, the linear alkylbenzene product 12 is isolated and exits the apparatus 10. It is noted that such separation processes are not necessary in all embodiments in order to isolate the alkylbenzene product 12. For instance, the alkylbenzene product 12 may be desired to have a wide range of carbon chain lengths and not require any fractionation to eliminate carbon chains longer than desired, i.e., heavies, or carbon chains shorter than desired, i.e., lights. Further, the fractionation performed at separator 16 may be sufficient such that no further fractionation is necessary despite the desired chain length range.

In certain embodiments, the natural oil source is castor, and the feed 13 comprises castor oils. Castor oils consist essentially of C₁₈ fatty acids with an additional, internal hydroxyl groups at the carbon-12 position. During fat splitting of a feed 13 comprising castor oil, it has been found that some portion of the carbon chains are cleaved at the carbon-12 position. Thus, deoxygenation creates a group of lighter C₁₀ to C₁₁ chains resulting and a group of non-cleaved heavier C₁₇ to C₁₈ chains. The first portion of fatty chains 18 may be rich in the lighter chains and the second portion of fatty chains 19 may be rich in the heavier chains. It should be noted that while castor oil is shown as an example of an oil with an additional internal hydroxyl group, others may exist. Also, it may be desirable to engineer genetically modified organisms to produce such oils by design. As such, any oil with an internal hydroxyl group may be a desirable feed oil.

The second portion of fatty chains 19 is not optimal for forming linear alkylbenzene. Thus, the stream of second portion of fatty chains 19 formed by the separator 16 are utilized herein to produce a different commercially valuable and renewable stream. As a result, utilization of the feed 13 is maximized.

As shown in FIG. 1, the second portion of fatty chains 19 is fed to an oleochemical production apparatus 80 for producing the oleochemical product 12, such as esters, alcohols, alkoxylates, ether sulfates, ether phosphates, sulfosuccinates, and/or other oleochemicals. In an exemplary embodiment, the oleochemical production apparatus 80 includes units 82 and 84 for processing the second portion of fatty chains 19. While the oleochemical production apparatus 80 is illustrated as including two processing units 82 and 84, more or fewer processing units may be included in the oleochemical production apparatus 80. In an exemplary process, the second portion of fatty chains 19 is fed to an esterification unit 82. The esterification unit 82 forms fatty acid methyl esters that are then fed to a sulfonation unit 84. The sulfonation unit 84 forms a sulfo-fatty acid esters, such as methyl ester sulfonate, as the oleochemical product 12.

Typically, no further deoxygenation is needed in the oleochemical production apparatus 80. Rather, in the apparatus 80, the second portion of fatty chains 19 are processed as selected for the desired oleochemical product 12. For example, the second portion of fatty chains 19 may undergo esterification, sulfonation, amidation, ethoxylation, hydrogenation, sulfation, epoxidation, chlorination, conjugation, fractionation, distillation, hardening, bleaching and/or other processing to form the desired oleochemical product 12.

While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the subject matter in any way. Rather, the foregoing Detailed Description will provide those skilled in the art with a convenient road map for implementing an exemplary embodiment, it being understood that various changes may be made in the function and arrangement of elements described in an exemplary embodiment without departing from the scope as set forth in the appended Claims and their legal equivalents.

1. A method for co-production of an alkylbenzene product and an oleochemical product from a natural oil, the method comprising:
fat splitting the natural oil to form a stream of free fatty chains;
fractionating the stream of free fatty chains to separate a first portion of free fatty chains and a second portion of free fatty chains;
processing the first portion of free fatty chains to provide the alkylbenzene product by a method comprising deoxygenating the first portion of free fatty chains to produce normal paraffins;
dehydrogenating the normal paraffins to provide mono-olefins;
alkylating benzene with the mono-olefins under alkylation conditions to provide an alkylation effluent comprising alkylbenzenes and benzene; and
isolating the alkylbenzenes to provide the alkylbenzene product; and
processing the second portion of free fatty chains to form the oleochemical product by a method comprising performing an esterification process and a sulfonation process to form a methyl ester sulfonate product.

2. The method of claim 1 wherein fractionating the stream comprises separating C₁₀ to C₁₃ free fatty chains as the first portion of free fatty chains and C₁₄₊ free fatty chains as the second portion of free fatty chains.

3. The method of claim 1 wherein fractionating the stream comprises separating C₁₀ to C₁₃ free fatty chains as the first portion of free fatty chains, separating C₉₋ free fatty chains as the second portion of free fatty chains, and separating C₁₄₊ free fatty chains as a third portion of free fatty chains.

4. The method of claim 1 wherein fractionating the stream comprises separating C₁₀ to C₁₃ free fatty chains as the first portion of free fatty chains and wherein the first portion of free fatty chains comprises at least about 97 wt % C₁₀ to C₁₃ free fatty chains.

5. The method of claim 4 wherein fractionating the stream comprises separating C₁₀ to C₁₃ free fatty chains as the first portion of free fatty chains and wherein the first portion of free fatty chains comprises no more than about 2 wt % C₉₋ free fatty chains.

6. The method of claim 5 wherein fractionating the stream comprises separating C₁₀ to C₁₃ free fatty chains as the first portion of free fatty chains and wherein the first portion of free fatty chains comprises no more than about 1 wt % C₁₄₊ free fatty chains.

7. (canceled)

8. (canceled)

9. The method of claim 1 further comprising providing palm kernel oil or coconut oil as the natural oil.

10. The method of claim 1 wherein the natural oil comprises fatty acids with internal hydroxyl groups, and wherein deoxygenating the natural oil causes cleaving and provides the first portion of free fatty chains and the second portion of free fatty chains.

11. A method for co-production of an alkylbenzene product and an oleochemical product from natural oil source triglycerides comprising:
fat splitting the natural oil source triglycerides to form a stream comprising glycerol and fatty acids;
fractionating the stream to separate glycerol, a first portion of fatty acids and a second portion of fatty acids;
deoxygenating the first portion of fatty acids to form normal paraffins;
dehydrogenating the normal paraffins to provide mono-olefins;
alkylating benzene with the mono-olefins under alkylation conditions to provide an alkylation effluent comprising alkylbenzenes and benzene;
isolating the alkylbenzenes to provide the alkylbenzene product; and
processing the second portion of fatty acids to form the oleochemical product by a method comprising performing an esterification process and a sulfonation process to form a methyl ester sulfonate product.

12. The method of claim 11 wherein fractionating the stream comprises separating C₁₀ to C₁₃ fatty acids as the first portion of fatty acids and C₁₄₊ fatty acids as the second portion of fatty acids.

13. The method of claim 11 wherein fractionating the stream comprises separating C₁₀ to C₁₃ fatty acids as the first portion of fatty acids and C₉− fatty acids and C₁₄₊ fatty acids as the second portion of fatty acids.

14. The method of claim 11 wherein fractionating the stream comprises separating C₁₀ to C₁₃ fatty acids as the first portion of fatty acids and wherein the first portion of fatty acids comprises at least about 97 wt % C₁₀ to C₁₃ fatty acids.

15. The method of claim 14 wherein fractionating the stream comprises separating C₁₀ to C₁₃ fatty acids as the first portion of fatty acids and wherein the first portion of fatty acids comprises no more than about 2 wt % C₉− fatty acids.

16. The method of claim 15 wherein fractionating the stream comprises separating C₁₀ to C₁₃ fatty acids as the first portion of fatty acids and wherein the first portion of fatty acids comprises no more than about 1 wt % C₁₄₊ fatty acids.

17. (canceled)

18. A method for co-production of an alkylbenzene product and an oleochemical product from a natural oil comprising:
fat splitting the oil to form fatty acids;
fractionating the fatty acids to separate a first portion of fatty acids and a second portion of fatty acids;
deoxygenating a first portion of fatty acids with hydrogen to form a stream comprising paraffins;
dehydrogenating the paraffins to provide mono-olefins and hydrogen;
recycling the hydrogen to support deoxygenating the first portion of fatty acids;
alkylating benzene with the mono-olefins under alkylation conditions to provide an alkylation effluent comprising alkylbenzenes and benzene;
isolating the alkylbenzenes to provide the alkylbenzene product; and
processing a second portion of fatty acids to form the oleochemical product by a method comprising performing an esterification process and a sulfonation process to form a methyl ester sulfonate product.

19. The method of claim 18 wherein the first portion of fatty acids comprises C₁₀ to C₁₃ fatty acids.

20. The method of claim 18 wherein the first portion of fatty acids comprises at least about 97 wt % C₁₀ to C₁₃ fatty acids, no more than about 2 wt % C₉₋ fatty acids, and no more than about 1 wt % C₁₄₊ fatty acids.
